(19) United States
(12) Patent Application Publication
(10) Pub. No.: US 2026/0320357 A1
(43) Pub. Date: Oct. 15, 2026

(54) HEALTH ABNORMALITY DETECTION AND CONSULTATION SUPPORT SYSTEM, METHOD AND COMPUTER READABLE STORAGE MEDIUM

(71) Applicant: WellnessMind AI Ltd, Anaheim, CA (US)

(72) Inventors: Xiaozhong Liu, Worcester, MA (US); Zhuoren Jiang, Hangzhou (CN)

(21) Appl. No.: 19/178,319

(22) Filed: Apr. 14, 2025

(51) Int. Cl. G06N 3/0475; G06N 3/047; G06N 3/096; G16H 50/30

(52) U.S. Cl. CPC G06N 3/0475; G06N 3/047; G06N 3/096; G16H 50/30

(57) ABSTRACT

Disclosed is a health abnormality detection and consultation support system, method and computer readable storage medium, integrating with a generative large language model. The support system includes a multi-physiological signal data collection module, an abnormality detection module, a generative large language model interaction module, a user feedback and joint learning module, and a risk assessment and alert module.

### TECHNICAL FIELD

The present disclosure relates to a health abnormality detection and consultation support system, method and computer readable storage medium, and more particularly to a health abnormality detection and consultation support system, method and computer readable storage medium integrated with a generative large language model.

### BACKGROUND

Existing wearable devices are mostly based on single physiological signals to detect health abnormalities, and are unable to cope with the multi-dimensional detection needs of complex symptoms. For example, diseases such as heart failure may be manifested in the early stage as a combination of heart rate fluctuations, abnormalities in blood pressure and blood oxygen, as well as subjective symptoms such as dizziness and fatigue, which are difficult to be comprehensively captured, fully recognized, and integrated by a single signal. In addition, existing methods may not be capable of capturing temporal changes in health signals and processing dynamic features such as time-series patterns, making it difficult to accurately recognize early signs of complex symptoms. Meanwhile, existing systems generally rely on rule-based detection algorithms such as fixed thresholds, which is difficult to adapt to individual health requirements and fail to adjust detection strategies in real time according to dynamical health states. More importantly, such kinds of system have significant deficiencies in the ability to comprehensively analyze multi-modal health data and personalized abnormality identification, limiting the ability to respond to complex health problems.

On the other hand, although generative large language model has been initially applied in the field of health consultation, most of the existing systems are limited to a single-round question-and-answer interaction mode, and lack the ability of dynamic interaction and the integration mechanism of user feedback. Especially after abnormality detection, it fails to make full use of real-time data from wearable devices to generate dynamic multi-round consultation content, and difficult to optimize the detection results or adjust the consultation strategy by combining the user's subjective feedback, which restricts the accuracy and personalization of the consultation.

### SUMMARY

The present disclosure provides a health abnormality detection and consultation support system, method and computer readable storage medium, which is integrated with a generative large language model, aiming to overcome the deficiencies existing in the prior art, especially the deficiencies in the multi-modal data processing, the dynamic interaction and personalized abnormality judgment. The present disclosure could realize the fusion of multi-modal data of the wearable devices and the health abnormality detection and dynamic consultation support of the generative large language model, and can significantly enhance the intelligence level and the practical value of health monitoring.

The present disclosure generally relates to a health abnormality detection and consultation support system, integrating with a generative large language model, wherein the support system includes a multi-physiological signal data collection module, an abnormality detection module, a generative large language model interaction module, a user feedback and joint learning module, and a risk assessment and alert module; the multi-physiological signal data collection module collects multiple physiological signal data through a wearable device, the data is processed in a deep neural network model and the processed data is input to the abnormality detection module to perform abnormality detection; the abnormality detection module generates multi-physiological signal embedding vector, performs abnormality detection through an encoder, and outputs abnormality categories and severity explanations; the generative large language model interaction module receives abnormality categories and severity explanations output from the abnormality detection module, generates personalized inquiries by means of LoRA Fine-Tuning and RAG mechanisms, and generates targeted questions based on user's historical health information and knowledge base information; the user feedback and joint learning module combines physiological signal embedding vector and user feedback embedding to generate multi-modal abnormality judgments based on a cross-attention mechanism; the risk assessment and alert module generates personalized alert based on user feedback and the detection result of abnormality judgments.

In certain embodiment, the abnormality detection module dynamically optimizes an abnormality detection process by combining a time series abnormality detection algorithm with a multi-modal embedding representation, comprises the following steps: (1) normalizing different physiological signals and inputting to the encoder; (2) the encoder performing representation learning of multi-modal physiological signals based on a deep neural network model, and generating multi-modal embedding vector; (3) analyzing the embedding vector using a time series abnormality detection algorithm; (4) using a trained abnormality detection model for initial abnormality detection based on multi-modal embedding vector, a classifier model calculating the probability distribution of each abnormality category by a multi-layer neural network, and the system using a language generation model to generate the detection results to a natural language textual explanation and passing to the generative large language model interaction module.

Preferably, in step (2), let X={x₁, x₂, . . . xᵢ, . . . xₙ} be real-time collected physiological signal data, wherein xᵢ denotes the ith physiological signals; each signal xᵢ is converted into a feature vector eᵢ=Encoder(xᵢ) by an encoder, and the feature vector are combined into a multi-modal embedding vector E=[e₁, e₂, . . . , eₙ] as an input for the system abnormality detection.

Preferably, in step (4), let the abnormality category be labeled as y, the classifier outputs a probability distribution for each abnormality category as

P(y|E)=Softmax(W·E+b)

where W and b represent the weight matrix and bias vector of the classifier, respectively, the system preliminarily outputs the abnormality categories and severity scores based on P(y|E), generates textual explanation and passes to the generative large language model interaction module; P(y|E) is the probability distribution of abnormality categories calculated by W, b, and E, which denotes the likelihood of each abnormality category and is determined by the category y which is corresponded to the maximum of the probability distribution P(y|E).

In certain embodiment, generating process of the textual explanation includes: extracting features of the input time series data by a deep neural network model to generate a fixed-size feature vector representing the current health state; generating a natural language textual explanation based on P(y|E) for passing to the generative large language model interaction module.

Preferably, in the generative large language model interaction module, the generative large language model achieves updating in a health consultation task through LoRA Fine-Tuning, and generates personalized consultation questions by combining a dynamic Prompt optimization strategy based on the retrieval-generation mechanism of RAG; upon receiving the text from the abnormality detection module, the system uses the RAG mechanism to generate personalized inquiries; the RAG retrieves medical information related to abnormality detection from a dynamic medical knowledge base and combines with the user's historical health data to generate targeted sequences of questions; the model inputs a combination of the multi-modal embedding vector and the embedding vector of the content retrieved from the knowledge base into an autoregressive generative model to generate personalized inquiries.

Preferably, in the user feedback and joint learning module, the feedback text generates an embedding F=Model(t), wherein t is the feedback text; the system inputs E and F into a joint learning model and generates a joint embedding Z through a cross-attention mechanism; the system uses a classifier to determine the abnormality category and score in joint embedding Z to ensure the accuracy of the abnormality detection.

Another aspect, this disclosure also relates to a health abnormality detection and consultation method, comprising the following steps: collecting multiple physiological signal data through a wearable device, processing collected data in a deep neural network model; generating multi-physiological signal embedding vector based on collected data through an encoder and performing abnormality detection, outputting abnormality categories and severity explanations; based on the output abnormality categories and severity explanations, generating personalized inquiries by means of LoRA Fine-Tuning and RAG mechanisms, and generating targeted questions based on user's historical health information and knowledge base information; combing physiological signal embedding vector and user feedback embedding to generate multi-modal abnormality judgments based on a cross-attention mechanism; generating personalized alert based on user feedback and detection result of abnormality judgments.

Preferably, in the abnormality detection process, by combining a time series abnormality detection algorithm with a multi-modal embedding representation, dynamical optimization is performed, comprising the following steps: (1) normalizing different physiological signals and inputs to the encoder; (2) the encoder performing representation learning of multi-modal physiological signals based on a deep neural network model, and generating multi-modal embedding vector; (3) analyzing the embedding vector using a time series abnormality detection algorithm; (4) using a trained abnormality detection model for initial abnormality detection based on multi-modal embedding vector, a classifier model calculating the probability distribution of each abnormality category by a multi-layer neural network; and (5) using a language generation model to generate detection results to a natural language textual explanation.

Preferably, in the generating targeted questions process, updating in a health consultation task is achieved through LoRA Fine-Tuning; upon receiving the text of abnormality detection module result, the method uses the RAG mechanism to generate personalized inquiries; the RAG retrieves medical information related to abnormality detection from a dynamic medical knowledge base and combines with the user's historical health data to generate targeted sequences of questions; the method inputs a combination of the multi-modal embedding vector and the embedding vector of the content retrieved from the knowledge base into an autoregressive generative model to generate personalized inquiries.

Yet another aspect, this disclosure also relates to a computer readable storage medium storing a program that enables a computer to execute the health abnormality detection and consultation method as described above.

The present disclosure provides a health abnormality detection and dynamic consultation support system, which is based on multi-modal data fusion, generative large language model interaction and user feedback fusion, integrating the functions of physiological signal data collection, abnormality detection, personalized interactive consultation and risk alert. The system defined in the present disclosure is applicable to scenarios such as real-time health monitoring and personalized health management. Specifically, the system utilizes a time-series abnormality detection algorithm combined with multi-modal embedding to dynamically adjust the abnormality detection strategy and ensure the synergistic analysis capability of multi-modal data, especially in terms of time-series pattern capture and signal synergistic detection, to achieve accurate identification of early signs of complex symptoms. Through LoRA Fine-Tuning and based on the dynamic knowledge retrieval and Prompt optimization strategy of the RAG mechanism, multi-round of consultation questions are generated by combining medical knowledge and user personalized data to ensure the relevance of the questions and the accuracy of the diagnostic suggestions. The accuracy and precision of abnormality detection are significantly improved by combining multi-physiological signal data embedding with user feedback embedding through a joint learning model, and using the cross-attention mechanism to generate joint embeddings for determining abnormality categories and grades.

### BRIEF DESCRIPTION OF THE DRAWINGS

FIG. 1 is a block diagram illustrating the support system of the present disclosure.

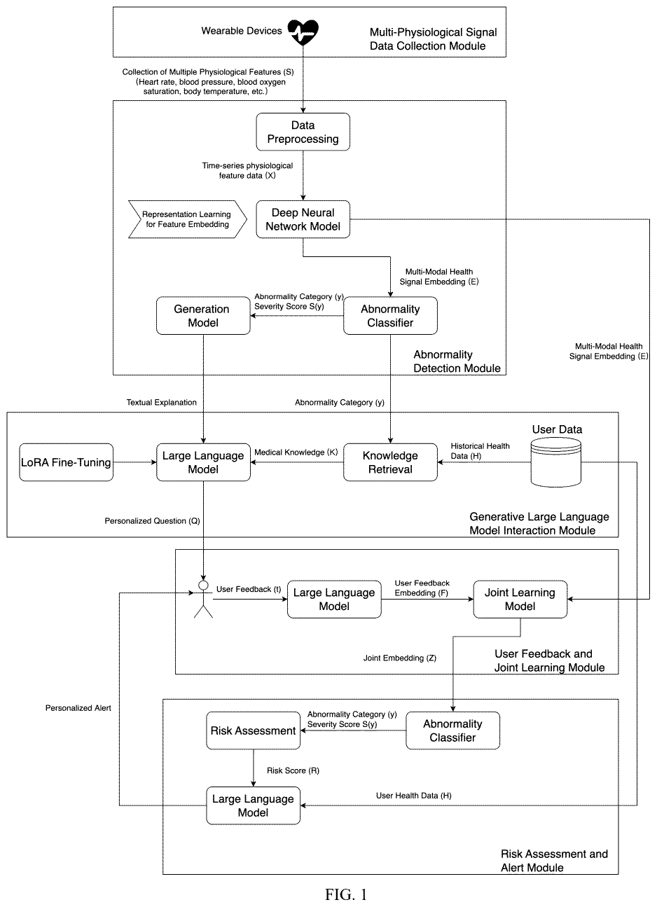

### DETAILED DESCRIPTION

In order to make the objectives, technical solutions and advantages of the present disclosure clearer, the embodiments of the present disclosure will be described below in detail with reference to the accompanying drawing.

The present disclosure provides a health abnormality detection and consultation support system. The system monitors the health status in real time and generates personalized consultation questions through a multi-modal deep neural network model and a generative large language model, assisting the user to actively identify potential health risks. The present disclosure is particularly suitable for health management of various chronic diseases, as well as remote medical consultation and other disclosure scenarios.

The health abnormality detection and consultation support system integrated with a generative large language model according to the present disclosure includes a multi-physiological signal data collection module, an abnormality detection module, a generative large language model interaction module, a user feedback and joint learning module, and a risk assessment and alert module. The input data of the system includes, but is not limited to, a plurality of physiological signals and user feedback data, the feedback data may be text, audio, or other forms of digitized information. The abnormality detection module generates multi-modal health signal embedding vector and performs abnormality detection via an encoder, outputting abnormality categories and severity explanations. The generative large language model interaction module receives the abnormality categories and severity explanations output from the abnormality detection module, generates personalized inquiries using LoRA Fine-Tuning and RAG mechanisms, and generates targeted questions based on user's historical health information and knowledge base information. The user feedback and joint learning module combines physiological signal embedding vector and user feedback embedding to generate multi-modal abnormality judgments based on the cross-attention mechanism. The risk assessment and alert module generates personalized alerts and health advice based on the detection results of abnormality judgments and user feedback. The system achieves risk grading based on the category and severity of the detected anomalies by means of a rule threshold and a dynamic learning method, and supports the dynamic adjustment of personalized health intervention strategies. The described system can be deployed on different hardware platforms, including but not limited to wearable devices, smartphones, cloud servers, and local computing devices. The form of user feedback includes, but is not limited to, text input, audio input, or other digitized information.

A health abnormality detection and consultation method according to the present disclosure, comprising the following steps:
collecting multiple physiological signal data through a wearable device, processing collected data in a deep neural network model;
generating multi-physiological signal embedding vector based on collected data through an encoder and performing abnormality detection, outputting abnormality categories and severity explanations;
based on the output abnormality categories and severity explanations, generating personalized inquiries by means of LoRA Fine-Tuning and RAG mechanisms, and generating targeted questions based on user's historical health information and knowledge base information;
combing physiological signal embedding vector and user feedback embedding to generate multi-modal abnormality judgments based on a cross-attention mechanism;
generating personalized alert based on user feedback and detection result of abnormality judgments.

Wherein in the abnormality detection process, by combining a time series abnormality detection algorithm with a multi-modal embedding representation, dynamical optimization is performed, comprises the following steps:
(1) normalizing different physiological signals and inputs to the encoder;
(2) the encoder performing representation learning of multi-modal physiological signals based on a deep neural network model, and generating multi-modal embedding vector;
(3) analyzing the embedding vector using a time series abnormality detection algorithm;
(4) using a trained abnormality detection model for initial abnormality detection based on multi-modal embedding vector, a classifier model calculating the probability distribution of each abnormality category by a multi-layer neural network; and
(5) using a language generation model to generate detection results to a natural language textual explanation.

Wherein in the generating targeted questions process, updating in a health consultation task is achieved through LoRA Fine-Tuning; upon receiving the text of abnormality detection module result, the method uses the RAG mechanism to generate personalized inquiries; the RAG retrieves medical information related to abnormality detection from a dynamic medical knowledge base and combines with the user's historical health data to generate targeted sequences of questions; the method inputs a combination of the multi-modal embedding vector and the embedding vector of the content retrieved from the knowledge base into an autoregressive generative model to generate personalized inquiries.

This disclosure also relates to a computer readable storage medium storing a program that enables a computer to execute the health abnormality detection and consultation method as described above.

### Multi-Physiological Signal Data Collection Module

A wide range of physiological signal data, including but not limited to heart rate, blood pressure, blood oxygen, body temperature, etc., are collected through wearable devices and uploaded to the system server in real time. The collected data is processed in a deep neural network model, the data is processed in a deep neural network model and the processed data is input to the abnormality detection module to perform abnormality detection.

### Abnormality Detection Module

The system utilizes a time series abnormality detection algorithm combined with multi-modal embedding to dynamically adjust the abnormality detection strategy and ensure the ability of synergistic analysis of multi-modal data, especially in the area of time series pattern capture and synergistic detection of signals, to achieve accurate identification of early signs of complex symptoms. The abnormality detection module dynamically optimizes the abnormality detection process by combining the time series abnormality detection algorithm with the multi-modal embedding representation. Specifically, the abnormality detection process includes the following steps:

### 1. Data Preprocessing

The system normalizes different physiological signals and inputs to the encoder. Let X={x₁, x₂, . . . , xᵢ, . . . , xₙ} be the real-time collected physiological signal data, where xᵢ denotes the ith physiological signal.

### 2. Representation Learning and Embedding Generation

The encoder generates multi-modal health signal embedding based on representation learning of multi-modal physiological signals from deep neural network models including, but not limited to, sequential models such as LSTM and Transformer. Representation learning enables the model to automatically extract key features of different physiological signals, thus effectively representing the time-dependent and interactive properties of the physiological signal data. Each signal xᵢ is converted into a feature vector eᵢ=Encoder(xᵢ) by the encoder. These feature vector are combined into multi-modal embedding vector E=[e₁, e₂, . . . , eₙ] as the input for system abnormality detection.

Let the parameter of the encoder be θ. The embedding of each physiological signal data xᵢ is:
eᵢ=Encoderθ(xᵢ)

All of the physiological signal data embedding are combined to form a multi-modal embedding vector E=[e₁, e₂, . . . , eₙ].

### 3. Dynamic Time Series Detecting

Embedding vector are analyzed using an improved time series abnormality detection algorithm, such as a gated recurrent unit-based GRU variant network. Specifically, includes: learning the time-series patterns of each physiological signal to capture dynamic trends; dynamically adjusting the abnormality detection thresholds for each signal, and coordinating the synergistic detection capability among multi-modal data through an adaptive weighting mechanism.

### 4. Abnormality Classifying and Representation Learning Output

The system performs initial abnormality detection based on the multi-modal embedding vector E using a trained abnormality detection model. The classifier model calculates the probability distribution P(y|E) for each abnormality category through a multi-layer neural network, where y denotes different abnormality categories. This probability distribution is used to determine the abnormality category and severity score. The classification network can be flexibly adapted according to the requirements, including but not limited to Fully Connected Layers and Softmax Layers, for generating the probability distribution and classifying the abnormalities based on the threshold. In addition, the system utilizes a language generation model to generate a natural language text explanation of the detection results, such as “heart rate abnormality detected, suspected cardiac abnormality” and passes it as a prompt to the generative large language model interaction module.

Let the abnormality category labels be y. The classifier outputs a probability distribution for each abnormality category:

P(y|E)=Softmax(W [symbol] +b)

where W and b represent the weight matrix and bias vector of the classifier, respectively, and Softmax is an activation function that could compress a K-dimensional real vector into another K-dimensional real vector so that each element is in the range of (0,1) and sums to 1, forming a probability distribution. The system preliminarily outputs the abnormality category and severity score based on P(y|E) and generates a textual explanation, and passes the same to the generative large language model interaction module.

In the abnormality detection module, the formula P(y|E)=Softmax(W·E+b) is the core formula used to generate the prediction of the abnormality categories. The weight matrix W is obtained by model learning on multi-modal physiological signals training data, which includes the mapping between the labeled abnormality categories and the multi-modal embedded features. b is the bias vector of the classifier, which is used to adjust the base probability of each abnormality category.

In the present disclosure, these parameters correspond to the specific parameters and data in the present disclosure as follows:

Wherein, the weight matrix W stores the mapping relationship between the multi-modal feature embedding E to the abnormality category y. In the present disclosure, it can be understood as a quantitative representation of the importance of the model's features for different abnormality categories. W corresponds to the weights learned by the classifier from the training data, which reflect the discriminating ability of different physiological signal features to different categories of abnormalities. For example, for heart rate data, a high heart rate may be more skewed towards cardiovascular abnormalities, whereas a low heart rate may be skewed towards drug response; for blood oxygen data, a low blood oxygen signature may directly correspond to respiratory abnormalities.

Multi-modal embedding vector E in Eq. is the core input, and is critical to understand how it interacts with W and b. E is a high-dimensional feature representation extracted from multi-modal physiological signals such as heart rate, blood oxygen, blood pressure, and body temperature after an embedding generative model, which serves as an input to the classifier. In the present disclosure, E corresponds to the embedded representation of the user's current physiological signals, reflecting the combined features of the multi-modal physiological signals. The generation process of E includes: feature extraction of the input time series data by a deep neural network model such as Transformer or RNN; generation of a fixed-size feature vector representing the current health state. P(y|E) is a probability distribution of the abnormality categories calculated by the system based on W, b, and E, and represents the likelihood of each abnormality category, which is determined by the category y corresponding to the maximum value of the probability distribution P(y|E). For example, if y denotes a cardiovascular abnormality and its probability is the maximum value, the initial decision of the abnormality category is a cardiovascular problem. This can be directly quantified based on the probability value; the higher the probability value is, the clearer the abnormality and the higher the severity score are. Based on P(y|E), the system generates a natural language text explanation for passing to the generative large language model interaction module.

### Generative Large Language Model Interaction Module

The generative large language model achieves efficient updating in the health consultation task through LoRA Fine-Tuning, and generates personalized consultation questions by combining dynamic Prompt optimization strategies based on the retrieval and generation mechanism of RAG. Upon receiving the text prompt from the abnormality detection module, the system uses the RAG mechanism to generate personalized inquiries. The RAG retrieves medical information related to abnormality detection from a dynamic medical knowledge base and combines with the user's historical health data to generate a sequence of targeted inquiries. Assuming that the content retrieved from the knowledge base is K={k₁, k₂, . . . , kₘ}, the model generates personalized inquiries by combining the inputs of E and the embedding vector K of the content retrieved from the knowledge base into an autoregressive generative model.

Generative large language model is fine-tuned by LoRA (Low-Rank Adaptation) to specifically optimize the adaptation of the health consultation task while significantly reducing the computational resources required for full parameter updating. LoRA Fine-Tuning allows the model to accomplish efficient parameter updating without changing the large-scale weights, by updating W′=W+A×B through low-rank matrices, where A and B are trainable low-rank matrices. LoRA is a technique for efficiently Fine-Tuning generative large language model to accomplish task-specific parameter optimization by introducing low-rank matrices A and B without modifying the weights W of large-scale pre-trained models. This approach is suitable for resource-constrained scenarios, such as health consultation tasks that require fast adaptation to different users or scenarios without the need for expensive full parameter updates to the entire model. The implementation is as follows:

LoRA Fine-Tuning: the model fine-tunes the weights using the low-rank matrices A and B to reduce computational complexity, and the Fine-Tuning adapts the model to the multi-modal health consultation task and improves the model's adaptability in responding to diverse inquiries. W denotes the weight matrix for the pre-trained generative large language model. These weights contain generalized linguistic knowledge and generative capabilities learned by the model from large-scale textual data. Matrices A denotes task-relevant tuning features for capturing specific information in the health consultation task. For example, how to generate reasonable questions or suggestions based on the user's health data. Matrices B denotes weight-varying filtering features that are used to identify and enhance vector that are relevant to the health consultation task while removing irrelevant noise features. By introducing A and B, the system can be efficiently fine-tuned for the health consultation task without the need to massively update W, thus saving computational resources and improving task adaptability. The core of this design is that, W remains fixed and still represents the generalized linguistic capabilities of the model, while A×B are key adjustments for generating specific content for the health consultation task. The LoRA Fine-Tuning method of the present disclosure achieves rapid adaptation of the model to specific task scenarios, such as generating personalized multi-round consultation questions, by using a specific health consultation task dataset for training and leveraging key features of the consultation task, such as multi-modal data, and personalized consultation needs. The Fine-Tuning method can quickly respond to dynamic multi-modal health data inputs, such as heart rate, blood pressure, and user's subjective feedback collected in real-time, and generate highly accurate consultation suggestions. This dynamic adaptation capability significantly improves the practicality of the model in complex health management scenarios.

RAG mechanism generation: the system retrieves the medical knowledge related to the abnormality detection results from the dynamic medical knowledge base and generates the knowledge set K={k₁, k₂, . . . , kₘ}. The retrieval process combines the abnormality categories and the user's historical health data to ensure the accuracy of knowledge retrieval. The system dynamically generates consultation questions by combining the abnormality detection results embedding vector E and the retrieved knowledge set K.

### User Feedback and Joint Learning Module

The joint learning model combines multi-physiological signal data embedding with user feedback embedding vector and uses the cross-attention mechanism to generate joint embedding to judge the type and level of abnormality, which significantly improves the accuracy and precision of abnormality detection. The user feedback and joint learning module is used to jointly judge the anomalies and do weight optimization.

User feedback generates the feedback embedding F=Model(t) by generative large language model, where t is the feedback text. The generated multi-modal embedding E and feedback embedding F are input into the joint learning model to generate the joint embedding Z through the cross-attention mechanism for deep information fusion, wherein Z=JointLearning(E,F). The system determines the type and level of anomalies in Z through a classifier to ensure the accuracy of the final abnormality detection. The joint learning combines the multi-modal features with the feedback embedding through the multi-layer network structure and adaptive weighting mechanism to improve the accuracy and personalization of the abnormality judgment. The system performs secondary judgment of abnormality category and abnormality level in this module. The joint learning model further combines user feedback information on the basis of the first abnormality detection to reassess the anomalies. The joint learning model applies the cross-attention mechanism to generate the joint embedding vector and passes to the classifier to generate the final abnormality judgment. The joint embedding not only characterizes the temporal changes and interactions of the physiological signals, but also integrates the user's subjective feedback to achieve a more accurate classification and severity assessment of the health state, improving the accuracy and personalization of the diagnosis.

### Risk Assessment and Alert Module

Based on the results of multi-modal detection and joint learning, the system determines the severity score of the abnormality through a probability threshold. Combined with the user's historical health data and knowledge base information, the system dynamically adjusts the alert content. Based on the risk score, the system provides a full range of alert services from healthy life guidance to emergency medical reminders, adapting to chronic disease management and acute disease diagnosis and treatment scenarios. For example, in chronic disease management scenarios, the system can advise users to undergo regular medical checkups or adjust lifestyle habits according to the risk score; in acute health risks, the system provides immediate medical advice or contact emergency ambulance services. Through this dynamic and personalized alert strategy, the system effectively improves the timeliness and accuracy of health management.

The present disclosure integrates wearable devices, multimodal data fusion technology and generative large language model, and innovatively realizes real-time monitoring and dynamic diagnosis of health abnormalities. With deep learning as the core, combined with multi-modal data processing and intelligent interaction of generative models, the system provides personalized health risk assessment and accurate alert, and provides users with reliable and efficient support in health management and medical auxiliary diagnosis. The present disclosure not only supports the processing of existing multimodal sign data, but also enables the access and analysis of more types of data through modular design, such as text signal, voice feedback, etc. By introducing joint learning technology and low-rank fine-tuning methods, the system can quickly adapt to new tasks and scenarios, which helps to expand the application scope of health management and real-time monitoring.

Although the implementations of the present disclosure are described above, the contents descried are only implementations used to understand the present disclosure and not intended to limit the present disclosure. Without departing from the spirit and scope disclosed by the present disclosure, any person skilled in the art to which the disclosure belongs can make any modifications and alterations to the forms and details of implementation. However, the protection scope of the present disclosure shall still be subject to the scope defined by the appended claims.

What is claimed is:

1. A health abnormality detection and consultation support system, integrating with a generative large language model, wherein the support system includes a multi-physiological signal data collection module, an abnormality detection module, a generative large language model interaction module, a user feedback and joint learning module, and a risk assessment and alert module;
the multi-physiological signal data collection module collects multiple physiological signal data through a wearable device, the data is processed in a deep neural network model and the processed data is input to the abnormality detection module to perform abnormality detection;
the abnormality detection module generates multi-physiological signal embedding vector, performs abnormality detection through an encoder, and outputs abnormality categories and severity explanations;
the generative large language model interaction module receives abnormality categories and severity explanations output from the abnormality detection module, generates personalized inquiries by means of LoRA Fine-Tuning and RAG mechanisms, and generates targeted questions based on user's historical health information and knowledge base information;
the user feedback and joint learning module combines physiological signal embedding vector and user feedback embedding to generate multi-modal abnormality judgments based on a cross-attention mechanism;
the risk assessment and alert module generates personalized alert based on user feedback and the detection result of abnormality judgments.

2. The health abnormality detection and consultation support system according to claim 1, wherein the abnormality detection module dynamically optimizes an abnormality detection process by combining a time series abnormality detection algorithm with a multi-modal embedding representation, comprises the following steps:
(1) normalizing different physiological signals and inputting to the encoder;
(2) the encoder performing representation learning of multi-modal physiological signals based on a deep neural network model, and generating multi-modal embedding vector;
(3) analyzing the embedding vector using a time series abnormality detection algorithm;
(4) using a trained abnormality detection model for initial abnormality detection based on multi-modal embedding vector, a classifier model calculating the probability distribution of each abnormality category by a multi-layer neural network, and the system using a language generation model to generate the detection results to a natural language textual explanation and passing to the generative large language model interaction module.

3. The health abnormality detection and consultation support system according to claim 2, wherein in step (2), let X={x₁, x₂, . . . xᵢ, . . . xₙ} be real-time collected physiological signal data, wherein xᵢ denotes the ith physiological signals; each signal xᵢ is converted into a feature vector eᵢ=Encoder(xᵢ) by an encoder, and the feature vector are combined into a multi-modal embedding vector E=[e₁, e₂, . . . , eₙ] as an input for the system abnormality detection.

4. The health abnormality detection and consultation support system according to claim 3, wherein in step (4), let the abnormality category be labeled as y, the classifier outputs a probability distribution for each abnormality category as
P(y|E)=Softmax(W·E+b)
where W and b represent the weight matrix and bias vector of the classifier, respectively, the system preliminarily outputs the abnormality categories and severity scores based on P(y|E), generates textual explanation and passes to the generative large language model interaction module; P(y|E) is the probability distribution of abnormality categories calculated by W, b, and E, which denotes the likelihood of each abnormality category and is determined by the category y which is corresponded to the maximum of the probability distribution P(y|E).

5. The health abnormality detection and consultation support system according to claim 4, wherein generating process of the textual explanation includes:
extracting features of the input time series data by a deep neural network model to generate a fixed-size feature vector representing the current health state;
generating a natural language textual explanation based on P(y|E) for passing to the generative large language model interaction module.

6. The health abnormality detection and consultation support system according to claim 5, wherein in the generative large language model interaction module, the generative large language model achieves updating in a health consultation task through LoRA Fine-Tuning, and generates personalized consultation questions by combining a dynamic Prompt optimization strategy based on the retrieval-generation mechanism of RAG;
upon receiving the text from the abnormality detection module, the system uses the RAG mechanism to generate personalized inquiries; the RAG retrieves medical information related to abnormality detection from a dynamic medical knowledge base and combines with the user's historical health data to generate targeted sequences of questions; the model inputs a combination of the multi-modal embedding vector and the embedding vector of the content retrieved from the knowledge base into an autoregressive generative model to generate personalized inquiries.

7. The health abnormality detection and consultation support system according to claim 3, wherein in the user feedback and joint learning module, the feedback text generates an embedding F=Model(t), wherein t is the feedback text; the system inputs E and F into a joint learning model and generates a joint embedding Z through a cross-attention mechanism; the system uses a classifier to determine the abnormality category and score in joint embedding Z to ensure the accuracy of the abnormality detection.

8. A health abnormality detection and consultation method, integrating with a generative large language model, comprising the following steps:
collecting multiple physiological signal data through a wearable device, processing collected data in a deep neural network model;
generating multi-physiological signal embedding vector based on collected data through an encoder and performing abnormality detection, outputting abnormality categories and severity explanations;
based on the output abnormality categories and severity explanations, generating personalized inquiries by means of LoRA Fine-Tuning and RAG mechanisms, and generating targeted questions based on user's historical health information and knowledge base information;
combing physiological signal embedding vector and user feedback embedding to generate multi-modal abnormality judgments based on a cross-attention mechanism;
generating personalized alert based on user feedback and detection result of abnormality judgments.

9. The health abnormality detection and consultation method according to claim 8, wherein in the abnormality detection process, by combining a time series abnormality detection algorithm with a multi-modal embedding representation, dynamical optimization is performed, which comprises the following steps:
(1) normalizing different physiological signals and inputs to the encoder;
(2) the encoder performing representation learning of multi-modal physiological signals based on a deep neural network model, and generating multi-modal embedding vector;
(3) analyzing the embedding vector using a time series abnormality detection algorithm;
(4) using a trained abnormality detection model for initial abnormality detection based on multi-modal embedding vector, a classifier model calculating the probability distribution of each abnormality category by a multi-layer neural network; and
(5) using a language generation model to generate detection results to a natural language textual explanation.

10. The health abnormality detection and consultation method according to claim 8, wherein in the generating targeted questions process, updating in a health consultation task is achieved through LoRA Fine-Tuning;
upon receiving the text of abnormality detection module result, the method uses the RAG mechanism to generate personalized inquiries; the RAG retrieves medical information related to abnormality detection from a dynamic medical knowledge base and combines with the user's historical health data to generate targeted sequences of questions; the method inputs a combination of the multi-modal embedding vector and the embedding vector of the content retrieved from the knowledge base into an autoregressive generative model to generate personalized inquiries.

11. A computer readable storage medium storing a program that enables a computer to execute the health abnormality detection and consultation method according to claim 8.
